(19) United States
(12) Patent Application Publication
(10) Pub. No.: US 2006/0265062 A1
(43) Pub. Date: Nov. 23, 2006

(54) A Resorbable, Biocompatible Moulded Body and a Procedure for its Production

(75) Inventors: Georg Berger, Panketal (DE); Gert Neumann, Berlin (DE)

Correspondence Address: NORRIS, MCLAUGHLIN & MARCUS, P.A., 875 THIRD AVE, 18TH FLOOR, NEW YORK, NY, 10022, US

(73) Assignee: BAM BUNDESANSTALT FUER MATERIALFORSCHUNG UND-PRUEFUNG, Berlin (DE)

(21) Appl. No.: 11/383,795

(22) Filed: May 17, 2006

(30) Foreign Application Priority Data
May 19, 2005 (DE) 10 2005 024 296.0

(51) Int. Cl. A61F 2/02

(52) U.S. Cl. 623/11.11

(57) ABSTRACT

The invention relates to a resorbable, biocompatible moulded body and a process for its production. The moulded body is mechanically processable and comprises an organic component which is solid at <40° C., which is biocompatible, which forms networks in water, and which is resorbable in the bodies of mammals within 3 to 20 days, and a solid, inorganic chemical component which is distributed within it with a particle size of d₅₀ of 7-800 nm, which is resorbable in the bodies of mammals within 7 to 180 days. This is obtained by converting a sol consisting of Ca compounds with an acidic phosphorous acid ester, contaminating the sol with additional cations by adding 0 to 5 weight % alcoholate or carboxylate of Na, K, Zn, Mg or mixtures of these, mixing the sol with the dissolved organic component, spray-drying the mixture and compressing the granulate obtained into a moulded body.

### FIELD OF INVENTION

The invention relates to a resorbable, biocompatible moulded body and a procedure for its production.

### STATE OF THE ART

A procedure is known for producing moulded bodies from gelatine, and using them in surgical procedures such as hip joint implants as a resorbable seal for hollow bones against any blood or tissue residue which may enter. Further known areas of use whereby moulded bodies are used intracorporally are in dentistry and emergency surgery (accident surgery). The disadvantage with these areas of application is that the elasticity module of the material is very low, with the result that it is difficult to process and has an extremely short disintegration period, usually of less than 3 days.

Furthermore, it is known that crystalline calcium phosphate can be used together with proteins or protein hydrolysates, for example with gelatine, either in fluid or paste form as structured composites, in particular as a substance for oral or dental maintenance (DE 199 30 335 or DE 103 40 542 A1). The inorganic phosphates are here precipitated from aqueous solutions.

An auxiliary surgical aid in the form of fibres or thin, plastifiable small leaves made of gelatine and glycerine, with the ceramic hydroxyapatite which they contain, with grain sizes of 0.5-1.8 mm, is described in U.S. Pat. No. 5,292,349.

### OBJECT OF THE INVENTION

The object of the invention is to improve the stability and processability of biologically resorbable moulded bodies, to provide a corresponding moulded body, and thus at the same time to enable the unrestricted growth of the human's own tissue into the moulded body, with the simultaneous biological disintegration of the moulded body, whereby the period of disintegration should be adapted according to the progress of the formation of the human's own substance.

### SUMMARY OF THE INVENTION

A resorbable, biocompatible moulded body is provided according to the invention, comprising an organic component which is solid in a temperature range of below 40° C., which form networks in water, and which is resorbable in the bodies of mammals within 3 to 20 days, and a solid, inorganic chemical component which is distributed within it with a particle size of d₅₀ of 5-100 nm, which is resorbable in the bodies of mammals within 7 to 60 days, whereby the proportion of the inorganic component lies within the range of 10-90 weight %, the proportion of the organic component lies within the range of 90-10% weight in relation to the total weight of the moulded body, and the moulded body is mechanically processable and has a compression strength in the range of 5 to 35 N/mm².

### DETAILED DISCLOSURE OF THE INVENTION

The object of the invention is also a moulded body with the features described, which can be obtained by

(a) dissolving calcium compounds or calcium compounds and at least one further metal compound in one or more solvent/s, and

(b) partially or fully removing the water;

(c) reacting the sol obtained with an acidic phosphoric acid ester with formula (I) or (Ia) or with a mixture thereof 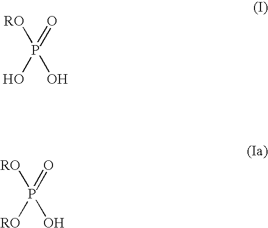
whereby R represents C₁-C₁₅ alkyl or aryl, and wherein R in formula (Ia) can be the same or different, and wherein the alkyl or aryl residues can be substituted by hydroxy and/or halogen; and
(d) contaminating the sol obtained with further cations by adding 0 to 5 weight % alcoholates or carboxylates of Na, K, Zn, Mg or mixtures of these
(e) mixing the fluid product of (d) with the organic component which is dissolved in water or an organic solvent or in a mixture of water and an organic solvent
(f) spray-drying the mixture of (e) with a viscosity suitable for spray drying of 1-3500 mPa·s at a temperature in the region of 80 to 160° C., and
(g) pressing the granulate obtained into moulded bodies using a pressure of between 1 and 100 kN.

Contaminating cations are preferably used with 0.1-5 weight %, in particular 0.5-3 weight %.

The moulded body according to the invention made of an organic component which is decomposable relatively rapidly and calcium phosphates which are chemically almost identical to the human body components and which are decomposable slightly more slowly, offers the opportunity of producing a temporary bone replacement with very good mechanical properties and a defined resorbability. This defined resorbability can be achieved by delaying the rapid decomposition of the organic component, if necessary using hydrophobisation, and the slightly slower ability to decompose of the organic component can be accelerated by selecting the particle size and the corresponding calcium compounds. In addition, the resorbability also depends on certain physical conditions in the site of application, such as the total mass to be resorbed, surfaces, individual specifics of the patient, etc.

Surprisingly, the initial strengths of the moulded body according to the invention exceed the initial strengths of the standard decomposable materials which are based on polyglycolates or on polylactides. Furthermore, in contrast to the above-mentioned, when the moulded body according to the invention is resorbed in the organism, no acidic decomposition products are created, which also represents a significant improvement as opposed to the known products. Articles have been published which conclude that acidic decomposition products encourage the activity of osteoclasts, i.e. unwanted bone decomposition.

Due to the hydrolytic stability of the organic component, there are no essential restrictions with regard to the handling of the material, thus making uncomplicated processing possible under normal air humidity conditions. The moulded body is therefore mechanically processable; i.e. it can be sawn, ground, drilled etc.

The very small particle size of the inorganic, non-crystalline or nanocrystalline component also enables those calcium compounds to be dissolved in contact with the tissue over a prolonged period of time which are otherwise difficult to dissolve, or which do not dissolve at all, and thus to replace the human body's own material in the specified form. When the percentage proportion of organic and inorganic components is produced by a specialist, also in relation to the conditions described above, relatively precise resorbability time periods can be set with an initially good processability of the moulded body.

Preferred resorbabilities lie in the range of 15-100 days; particularly preferred are 6-60 days, in particular 7-40 days.

Nanocrystalline hydroxyapatites (HA), β-tricalcium phosphate (TCP), calcium-alkali-orthophosphates such as Ca₂KNa(PO₄)₃ or Ca₁₀[K/NA] (PO₄)₇, calcium-alkali-metaphosphates or fluoroapatite (FA) is understood to refer to particle sizes in the region of 7 to 800 nm.

In the first stage in the production of the moulded body according to the invention, calcium compounds, e.g. inorganic salts or organic calcium compounds, are dissolved. The solvent can be water, an organic solvent or a mixture of there. In this way, calcium carboxylate can be dissolved in mixtures consisting of acetic acid and water together with alkali carboxylates, so that following the addition of the acidic phosphorous acid ester, nanoscale alkali-alkaline earth phosphates can be produced.

In general, the organic solvent can be a monovalent or polyvalent alcohol, acetic acid, acetyl acetone or a mixture thereof. A solvent consisting of a C₁-C₉ alkanol, glycol, glycerine or a mixture thereof is preferred.

Alongside these dissolved calcium compounds, further metal compounds can be included, such as those of sodium, potassium, magnesium, zinc or mixtures of these. Alcoholates and carboxylates consisting of sodium and potassium are preferred.

With this procedural step, solutions or suspensions are produced with particle sizes below 100 nm, which are here also referred to as nanoscale suspensions.

In cases when low PH values must be avoided in order to obtain a stable sol, organic complexing agents can be added to the solutions of nanoscale suspended inorganic salts as stabilisers. For example, it is possible with EDTA with mixtures consisting of calcium and alkali carboxylates in ethylene glycol to prevent precipitations of the components, even with high concentrations.

Special bioactive calcium phosphate ceramics with certain anions of the fluoroapatite type can be stabilised by adding salts of tertiary amines, preferably of triethanolamine.

In order to achieve a stabilisation of the inorganic salts which are dissolved in the organic solvents or which are nanoscale suspended, non-ionogenic tensides containing silicon can also be added.

Water is partially or fully removed from the nanoscale suspensions, for example via distillation, in order to achieve a sol. Advantageously, the distillation may be conducted in the presence of a second organic solvent, which is advantageously a monovalent C₄-C₉ alcohol, a polyvalent alcohol such as glycol, propylene glycol, butylene glycol or glycerine, or a mixture thereof, and which can be distilled out as azeotrope. Other solvents which form azeotropes with water, such as benzene, toluene or xylene, may also be used.

In the spirit of the invention, “water removal” means, alongside distilling out or drying measures, also the reaction with e.g. anhydrides or the toleration of low quantities of crystal water.

Essential for the invention is the removal or more or less water from the respective mixture of calcium compounds with the corresponding metal ions. This generally already leads to sols, of which the components are dissolved as nanoscale particles.

In the next procedural step, a phosphorous ester is added, which comprises at least one free OH group, and which is referred to as an “acidic” phosphorous ester. In this way, a durable calcium phosphate sol or calcium-Me-phosphate sol is obtained. If necessary, the sol must be left standing for a certain period of time, e.g. 2-20 hours, at room temperature (20-25° C.) or at an increased temperature (26-50° C.), until the viscosity suitable for the next procedural stage is reached. Here, a certain gel structure is formed, which may not however exceed a viscosity which is for the subsequent spray drying. A specialist will easily be able to monitor this using a GFA® gel time apparatus.

Preferred viscosities lie in the region of 1-3000 mPa·s, in particular 10-2000 mPa·s, measured with a rotation viscosimeter, at 25° C.

The acidic phosphorous acid ester is advantageously selected from the group consisting of a solution of phosphorous pentoxide in a C₁-C₁₆ alkanol, a solution of phosphorous pentoxide in a glycol which is optionally substituted by C₁-C₁₆ alkyl, hydroxyalkyl or halogen-alkyl, or a solution of phosphorous pentoxide in an aryl alkanol, which can also be substituted.

Particularly preferred as alkanols are propanol and butanol.

In the formulae (I) and (Ia) of the acidic phosphorous acid ester, the alkyl residue is preferably a C₁-C₄ alkyl residue, in particular a C₁-C₃ alkyl residue. The alkyl residue is preferably a phenyl or C₁-C₄ alkyl phenyl residue, in particular a phenyl- or C₁-C₃ alkyl phenyl residue, wherein alkyl corresponds to the named preferred denotations. Preferred substituents for the alkyl or alkyl residue are hydroxy, fluorine, chlorine or bromine.

Furthermore, hydrofluoric acid such as dehydrated HF can be added to the acidic phosphorous acid ester in cases when the ceramic sinter body to be produced should consist of, or contain, fluoroapatite. Chlorapatite can be produced in a similar manner.

According to a particular embodiment of the invention, the dry gel powder can be subjected to a calcination of up to 400° C., since usually, larger quantities of organic components still adhere to the powder. Up to this temperature, no crystalline transformation yet takes place.

The inorganic component is advantageously selected from the group consisting of nanocrystalline hydroxyapatite, nanocrystalline fluoroapatite, tricalcium phosphate, calcium potassium phosphate CaKPO₄, calcium sodium phosphate CaNaPO₄, mixtures of Ca—Na phosphate and Ca—K phosphate, and mixtures with diphosphates which contain calcium.

Particularly preferred are calcium potassium phosphate, calcium sodium phosphate, mixtures of Ca—Na phosphate and Ca—K phosphate, and their mixtures with diphosphates which contain calcium. All the phosphates and diphosphates named are non-crystalline substances according to x-ray diffraction.

The diphosphate is preferably Na₂CaP₂0₇, K₂CaP₂O₇, Ca₂P₂O₇ or a mixture thereof.

“Resorbable” in the spirit of the present invention means that essentially, no residues of the moulded body according to the invention originally introduced into the tissue or into the bone are present, and that this moulded body has been replaced at least by 95% by the human body's own material.

It is furthermore advantageous that the inorganic component contains a proportion of reactive phosphate groups of 10-50 mol %, preferably 20 mol %. In this way, particularly strong hydrogen bridge bonds are formed to the organic component.

In the moulded body according to the invention, the proportion of the inorganic component is preferably in the range of 60-85 weight %, in particular 65-85 weight %. Here, advantageously, 0.1-1.5% of other inorganic compounds may additionally be included, which allow a further adaptation of the inorganic component to the natural composition of the bone/cartilage or the blood, e.g. alkali chlorides, or compounds containing Mg, Zn or Si. The organic component then has a proportion of 40-10 weight %, in particular 35-15 weight %.

The organic component is a gelling agent based on gelatine, cellulose or polysaccharides. Particularly preferred for the organic component is gelatine, pectin or agar-agar, in particular gelatine. Mixtures of gelling agents may also be used.

With gelatine, the product gained from collagen can be used both under acidic conditions (isoelectric point in pH range 7.5-9.3), and in alkaline conditions (isoelectric point in pH range 4.7-5.2).

In a preferred embodiment of the invention, e.g. gelatine is acetylised prior to being mixed with the inorganic component, which causes the amino groups which are capable of forming hydrogen bridge bonds to be partially blocked, and therefore the solubility to be reduced. A similar process takes place when pectins are acetylised, when the solubility is reduced due to the formation of esters. Advantageously, the acetylisation is conducted with an acetic acid anhydride. Methylisation can also be conducted previously.

The object of the invention is also a process for producing a resorbable, biocompatible moulded body by

(a) dissolving calcium compounds or calcium compounds and at least one further metal compound in one or more solvent/s, and

(b) partially or fully removing the water

(c) converting the sol obtained with an acidic phosphorous acid ester of formula (I) or (Ia), or a mixture thereof 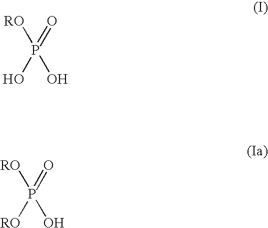
wherein R represents C₁-C₁₅ alkyl or aryl, and wherein R in formula (Ia) can be the same or different, and wherein the alkyl or aryl residues can be substituted by hydroxy and/or halogen, if necessary in the presence of HF or HCl, and
(d) contaminating the sol obtained with further cations through the addition or 0 to 5 weight % alcoholate or carboxylate of Na, K, Zn, Mg or mixtures of these
(e) mixing the fluid product of (d) with the organic component, which is dissolved in water or an organic solvent, or dissolved in a mixture of water/organic solvent
(f) spray-drying the mixture of (e) with a viscosity suitable for spray-drying in the region of 1-3500 mPa·s at a temperature in the range of 80 to 160° C., if necessary with the addition of pharmaceuticals, and
(g) compressing the granulate obtained into moulded bodies under a pressure of between 1 and 100 kN. Higher viscous suspensions can be spray-dryed by ultrasonic use at the same time.

In a particular embodiment, the mixture from step (e) is spray-dried and treated during the spray-drying with microwaves with a power of 500 W or above, in order to achieve a partial reaction between the inorganic and the organic component.

The addition of pharmaceuticals during the spray-drying, or—with temperature-sensitive pharmaceuticals—to the granulate following the spray-drying can be an advantageous measure within the framework of the invention.

The moulded body according to the invention is mechanically stable, can be processed using drilling and grinding, and has a compressive strength of 15-22 N/mm² (compression test according to ZWICK® apparatus BDO-FB005TS.

According to a further preferred embodiment of the invention the moulded body has a layered structure, with an outer layer which comprises 15-85 weight % of inorganic component and 15-25 weight % of organic component, and an inner section which comprises 15-25 weight % of inorganic component, and 75-80 weight % of organic component.

According to another preferred embodiment of the invention the moulded body comprises a concentration gradient of inorganic and organic components, in which the concentration of the inorganic component decreases from outside to inside, and the concentration of the organic component increases from inside to outside.

The production of preferred embodiments of this type can for example be achieved by pressing layers together which have different component concentrations.

The invention will now be described in greater detail with reference to examples and comparative examples. These contain all percentages and weight percentages.

### EXAMPLE 1

### Hydroxyapatite

30.7 g calcium acetate (200 mmol) is dissolved in 30 ml of acetic acid and 30 ml of water. In a further receptacle, in order to produce the “acidic” ester, 10.7 g of pure P₂O₅ is dissolved in 80 g of butanol. The (nanoscale) precipitation is then achieved by adding the “acidic” ester to the calcium acetate solution while thoroughly stirring.

Particle size: 40-100 nm.

5 g of gelatine is dissolved in 100 ml of hot water with a temperature of 90° C. To this solution is added 50 ml of an aqueous suspension of nanoscale hydroxy apatite with a solid content of 30 weight % and homogenised with ultrasound. At 150° C., this solution is then spray-dried (viscosity 1280 mPa·s). In order to create an optimum moisture level of the powder obtained of between 3 and 9%, it is dried again in a vacuum drying cabinet at 110° C. and 10 mbar for between one and two hours.

In order to produce the moulded bodies, 2 g of the powder is weighed in a tablet form (ø 10 mm) which is warmed to 60° C., and is compressed for 5 min at 25 kN.

After cooling, a moulded body with high stability is created, which also has a high level of flexibility. In the compression test, the cylinder deforms under pressures of approximately 20 N/mm² up to 10%, before finally being destroyed. Resorbability in animal experiments: 30-58 days.

### EXAMPLE 2

### Tricalcium Phosphate

18.6 g Calcium acetate (100 mmol) are dissolved in 100 ml acetic acid and 10 ml water at 40° C. To this solution a solution of 3 g gelatine in 40 ml water is added. The nanoscalic precipitation follows after addition of 7.7 g 85% phosphoric acid during powerful stirring by further addition of 100 ml 2-propanol approx. 30° C. after 24 h. After spray-drying at 150° C. an aggregated powder is received with primary particles <1 μm.

The production of moulded bodies is achieved by compression with the same result. A break-proof moulded body which can be mechanically processed very well using drilling, grinding etc. is obtained.

Resorbability in animal experiments: 44-48 days.

### EXAMPLE 3

### Calcium-Alkali-Orthophosphate Ca₂KNa(PO₄)₂

30 g Calcium acetate, 9.8 g potassium acetate, 13.6 g sodium acetate and 4.3 g magnesium acetate are dissolved in 100 ml water. For the nanoscalic precipitation 0.3 g hexadecyl trimethyl ammonium chloride as a tensid and a solution of 108.4 g acidic ester prepared from 2-propanol and phosphorous pentoxide are added for forming the gel at 25° C. The powder for compression is produced by spray-drying the suspension of the inorganic component with particle sizes of 50-95 nm together with acetylated gelatine (viscosity 2030 mPa·s). The moulded body obtained after compression is surface treated and used in animal experiments.

Resorbability: 28-35 days.

### EXAMPLE 4

### Calcium Metaphosphate Ca(PO₃)₂

18.6 g Calcium acetate (100 mmol) are dissolved in 100 ml acetic acid and 10 ml water at 40° C. The nanoscalic preparation takes place at approx. 30° C. after 24 h by addition of 23.1 g 85% phosphoric acid by powerful stirring and addition of 100 ml 2-propanol.

After spray-drying at 150° C. an aggregated powder is received which primary particles of the powder are <1 μm if 2% stearic acid are added to the 2-propanol. To receive a special quick resorbable composite powder 4 g hydroxyethylcellulose are dissolved in 100 ml water and added to the suspension of calcium phosphate. The spray-dried powder can be compressed in the above described manner to a moulded body. The nanoscalic metaphosphate is received by calcination of the powder prepared without hydroxyethylcellulose at 400° C.

### EXAMPLE 5

### Fluoroapatite

30.7 g Calcium acetate (200 mmol) are dissolved in 30 ml of acetic acid and 30 ml of water. For the purpose of forming the “acidic” ester, in a further receptacle, 10.7 g of pure P₂O₅ are dissolved in 80 g butanol. Furthermore, 2.0 g (40 mmol) of hydrofluoric acid with 7.0 g of acetic acid anhydride are dehydrated and added to the “acidic” ester. The (nanoscale) precipitation is then achieved by adding the “acidic” ester to the calcium acetate solution while stirring thoroughly.

Particle size: 60-100 nm.

5 g of gelatine are dissolved in 100 ml of hot water with a temperature of 90° C. To this solution is added 50 ml of an aqueous suspension of nanoscale fluorapatite with a solid content of 30 weight %, and homogenised with ultrasound. At 150° C., this solution is then spray-dried. In order to create an optimum moisture level of the powder obtained of between 3 and 9%, it is then dried again for between one and two hours in a vacuum drying cabinet at 110° C. and 10 mbar.

In order to produce the moulded bodies, 2 g of the powder are weighed in a tablet form (ø 10 mm) which is warmed to 60° C., and is compressed for 5 min at 25 kN.

After cooling, a moulded body with high stability is created, which also has a high level of flexibility. In the compression test, the cylinder deforms under pressures of approximately 20 N/mm² up to 10%, before finally being destroyed.

Resorbability in animal experiments: 48-72 days.

1. A resorbable, biocompatible moulded body, comprising a biocompatible organic component which is solid in a temperature range of below 40° C., which form networks in water, which is resorbable in the bodies of mammals for between 3 and 20 days, and a solid, inorganic chemical component which is distributed within it with a particle size d₅₀ of 7-800 nm, which is resorbable in the bodies of mammals within 7 to 180 days, whereby the proportion of the inorganic component constitutes at least 15 weight %, the proportion of the organic component constitutes 85 weight % at most in relation to the total weight of the moulded body, and the moulded body is mechanically processable and has a compression strength in the range of 5 to 35 N/mm².

2. A moulded body according to claim 1, wherein the proportion of the inorganic component lies in the range of 15-90 weight %, and the proportion of the organic component lies in the range of 10-85 weight %.

3. A moulded body according to claim 1, wherein the additional metal compound comprises sodium, potassium, magnesium or a mixture of these.

4. A moulded body according to claim 1, wherein the calcium compound is a calcium phosphate which contains an alkaline.

5. A moulded body according to claim 1, wherein the acidic phosphorous ester is selected from the group consisting of a solution of phosphorous pentoxide in a C₁-C₁₆ alkanol, a solution of phosphorous pentoxide in a C₁-C₁₆ alcoxylated glycol or a solution of phosphorous pentoxide in an aryl alkanol.

6. A moulded body according to claim 1, wherein the inorganic component is selected from the group consisting of nanocrystalline hydroxyapatite, nanocrystalline fluoroapatite, tricalcium phosphate, calcium potassium phosphate, calcium sodium phosphate, mixtures of Ca—Na phosphate and Ca—K phosphate, calcium metaphosphate, calcium-alkali metaphosphates and mixtures thereof with diphosphates which contain calcium.

7. A moulded body according to claim 6, wherein the diphosphate is Na₂CaP₂0₇, K₂CaP₂O₇, Ca₂P₂O₇ or a mixture thereof.

8. A moulded body according to claim 1, wherein the proportion of the inorganic component lies in the range of 60-90 weight %.

9. A moulded body according to claim 8, wherein the range is 65 to 85 weight %.

10. A moulded body according to claim 1, wherein the organic component comprises a gelling agent based on gelatine, cellulose or polysaccharides.

11. A moulded body according to claim 10, wherein the organic component is gelatine.

12. A process for producing a resorbable, biocompatible moulded body according to claim 1 by
(a) dissolving calcium compounds or calcium compounds and at least one further metal compound in one or more solvent/s, and
(b) partially or fully removing the water
(c) converting the sol obtained with an acidic phosphorous acid ester of formula (I) or (Ia), or a mixture of these 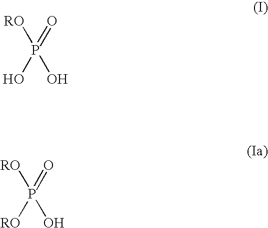
wherein R represents C₁-C₁₅ alkyl or aryl, and wherein R in formula (Ia) can be the same or different, and wherein the alkyl or aryl residue can be substituted by hydroxy, halogen or both, if necessary in the presence of HF or HCl, and
(d) contaminating the sol obtained with further cations through the addition or 0 to 5 weight % alcoholate or carboxylate of Na, K, Zn, Mg or mixtures of these
(e) mixing the fluid product of (d) with the organic component, which is dissolved in water or an organic solvent, or dissolved in a mixture of water/organic solvent
(f) spray-drying the mixture of (e) with a viscosity suitable for spray-drying in the range of 1-3500 mPa·s at a temperature in the range of 80 to 160° C., if necessary with the addition of pharmaceuticals, and
(g) compressing the granulate obtained into moulded bodies under a pressure of between 1 and 100 kN.

13. A process according to claim 12, wherein the mixture from step (e) is spray-dried and treated during the spray-drying with microwaves.

14. A process according to claim 12, wherein an acetylated or methylated gelatine is used as the organic component.

15. A process according to claim 12, wherein the removal of water according to step (b) via distillation is conducted in the presence of a second organic solvent.

16. A process according to claim 12, wherein as an organic component, one with a proportion of reactive phosphate groups of 10-50 mol % is used.
